(19) United States
(12) Patent Application Publication
(10) Pub. No.: US 2022/0023822 A1
(43) Pub. Date: Jan. 27, 2022

(54) NANOPORE FORMING METHOD AND ANALYSIS METHOD

(71) Applicant: Hitachi, Ltd., Chiyoda-ku, Tokyo (JP)

(72) Inventor: Itaru Yanagi, Tokyo (JP)

(21) Appl. No.: 17/297,132

(22) PCT Filed: Nov. 7, 2019
(86) PCT No.: PCT/JP2019/043577

§ 371 Date: May 26, 2021

(51) Int. Cl. B01J 19/08; G01N 27/00; B82Y 40/00; B82Y 35/00

(52) U.S. Cl. CPC B01J 19/08; B82Y 35/00; B82Y 40/00; G01N 27/002

(57) ABSTRACT

Provided is a technique for stably forming a single nanopore by dielectric breakdown for a membrane having a high dielectric breakdown withstand voltage. In the nanopore forming method of the present disclosure, a SiNx film is placed between the first aqueous solution and the second aqueous solution, the first electrode is brought into contact with the first aqueous solution, and the second electrode is brought into contact with the second aqueous solution, and a voltage is applied to the first electrode and the second electrode. The SiNx film has a composition ratio of 1<x<4/3. At least any one of the first aqueous solution and the second aqueous solution has the pH of 10 or more.

### TECHNICAL FIELD

The present disclosure relates to a nanopore forming method and an analysis method.

### BACKGROUND ART

As a means for detecting and analyzing molecules and particles existing in an aqueous solution, a technique using a nanopore device is being studied. In the nanopore device, the membrane is provided with a pore (nanopore) having approximately the same size as the molecules or particles to be detected, the chambers provided above and below the membrane are filled with an aqueous solution, and electrodes are provided to come into contact with the aqueous solutions in both chambers. An object to be detected is introduced on one side of the chambers, and is electrophoresed by applying a potential difference between the electrodes so as to pass through the nanopore. Thus, a time change of an ion current (blockade signal) flowing between both electrodes is measured, so that it is possible to detect that the object to be detected passes through or to analyze the structural characteristics of the object to be detected.

In the manufacturing of nanopore devices, a method of forming a nanopore by processing a semiconductor substrate or a semiconductor material by a semiconductor process has attracted attention because of its high mechanical strength and the like. As such a nanopore forming method, for example, in NPL 1, a silicon nitride film (SiNx film) is used as a membrane, and a TEM (transmission electron microscope) device is used to narrow down the irradiation area of the electron beam on the membrane, and energy or current is controlled, as a result, the nanopore having a diameter of 10 nm or less is formed.

Further, PTL 1 and NPLs 2 to 4 disclose nanopore forming methods utilizing dielectric breakdown of a membrane. In these methods, first, the upper and lower chambers sandwiching the non-perforated SiNx membrane are filled with an aqueous solution, the electrodes are immersed in the aqueous solution of each chamber, and a high voltage is continuously applied between the two electrodes. When the current value between the electrodes rises sharply (the membrane breaks down) and the current value reaches the preset cutoff current value, it is determined that nanopore has been formed, and the application of high voltage is stopped to form the nanopore. This nanopore forming method has the advantages of significantly reducing the manufacturing cost and improving the throughput as compared with the nanopore forming using the TEM device. Further, in this nanopore forming method, after forming the nanopore in the membrane, it is possible to shift to the measurement of the object to be detected without removing the membrane from the chamber. Therefore, there is an advantage that the nanopore is not exposed to pollutants in the atmosphere and noise during measurement is reduced.

One of the applications of measurement using a nanopore is decoding the base sequence of DNA (DNA sequencing). That is, it is a method of determining the sequence of four types of bases in a DNA strand by detecting a change in ionic current when DNA passes through a nanopore.

Another one of the applications of measurement using a nanopore is the detection and counting of a specific object in an aqueous solution. For example, NPL 5 discloses that PNA and PEG are bound only to DNA having a specific sequence existing in the aqueous solution, and the change in ion current as the PNA and PEG-modified DNA passes through a nanopore is measured, so that DNA having a specific sequence is detected and counted.

In such nanopore measurement, since the object is measured while applying a voltage to the membrane, it is desirable that the breakdown withstand voltage (hereinafter, referred to as dielectric breakdown withstand voltage) with respect to the voltage applied to the membrane is high. That is, in nanopore measurement, it is desirable to use a membrane that does not easily break down even if the voltage required for measurement is applied for a long time. It is also known that a high voltage is applied to the membrane due to static electricity generated during setup before measurement, and the membrane undergoes dielectric breakdown (NPL 6). In order to prevent such dielectric breakdown due to the high voltage derived from static electricity, it is desirable that the membrane has high dielectric breakdown withstand voltage. Therefore, a membrane that does not easily break down even when a high voltage is applied is desirable.

### CITATION LIST

### Patent Literature

PTL 1: WO 2013/167955

### Non-Patent Literature

NPL 1: Jacob K Rosenstein, et al., Nature Methods, Vol. 9, No. 5, 487-492 (2012)
NPL 2: Harold Kwok, et al., PloS ONE, Vol. 9, No. 3, e92880. (2013)
NPL 3: Kyle Briggs, et al., Nanotechnology, Vol. 26, 084004 (2015)
NPL 4: Kyle Briggs, et al., Small, 10(10):2077-86 (2014)
NPL 5: Trevor J. Morin, et al., PLoS ONE 11(5):e0154426. doi:10.1371/journal.pone.0154426 (2016)
NPL 6: Kazuma Matsui, et al., Japanese Journal of Applied Physics, Vol. 57, No. 4 (2018)
NPL 7: Itaru Yanagi, et al., Scientific Report, 8, 10129 (2018).

### SUMMARY OF INVENTION

### Technical Problem

However, even if an attempt has been made to form a nanopore using dielectric breakdown on a SiNx membrane (1<x<4/3) having a high dielectric breakdown withstand voltage by the methods of NPLs 2 to 4, it has been found that it is difficult to form a single nanopore.

Therefore, the present disclosure provides a technique for stably forming a single nanopore by dielectric breakdown for a membrane having a high dielectric breakdown withstand voltage.

### Solution to Problem

A nanopore forming method of the present disclosure includes, arranging a SiNx film between a first aqueous solution and a second aqueous solution, bringing a first electrode into contact with the first aqueous solution, and bringing a second electrode into contact with the second aqueous solution, and applying a voltage to the first electrode and the second electrode. The SiNx film has a composition ratio of 1<x<4/3. At least any one of the first aqueous solution and the second aqueous solution has a pH of 10 or more.

Other features of the disclosure will be clear from the description and the accompanying drawings of this specification. In addition, embodiments of the disclosure are achieved and realized by elements, combinations of various elements, the following detailed description, and the attached claims.

It is necessary to understand that the description of this specification is given only as a typical example, and does not limit the scope of claims or applications of the disclosure.

### Advantageous Effects of Invention

According to the present disclosure, a single nanopore can be stably formed by dielectric breakdown on a membrane having a high dielectric breakdown withstand voltage.

Objects, configurations, and effects besides the above description will be apparent through the explanation on the following embodiments.

### BRIEF DESCRIPTION OF DRAWINGS

FIGS. 1A and 1B are schematic diagrams illustrating a device configuration for a nanopore forming method according to a first embodiment.

FIGS. 2A and 2B are diagrams illustrating the results of a first comparative example.

FIGS. 3A and 3B are diagrams illustrating the results of a second comparative example.

FIGS. 4A and 4B are diagrams illustrating the results of a third comparative example.

FIGS. 5A and 5B are diagrams illustrating the results of a fourth comparative example.

FIGS. 6A and 6B are diagrams illustrating the results of a first example.

FIG. 7 is a diagram illustrating the results of a second experimental example.

FIGS. 8A-8C are diagrams illustrating the results of a third experimental example.

FIGS. 9A-9D are schematic diagrams illustrating a structure of a laminated film according to a second embodiment.

FIG. 10 is a diagram for explaining a voltage application method according to a third embodiment.

FIG. 11 is a diagram for explaining a voltage application method according to a fourth embodiment.

### DESCRIPTION OF EMBODIMENTS

In all the drawings for explaining embodiments, those having the same function are given the same reference numerals, and the repeated description thereof is omitted as much as possible. Hereinafter, embodiments of the present disclosure will be described on the basis of the drawings. The structure and materials of the device described in the embodiments are examples for embodying the idea of the present disclosure, and the present disclosure does not strictly specify the materials and dimensions. Further, the specific voltage value, current value, and voltage application time described in the embodiments are examples for embodying the idea of the present disclosure, and the present disclosure does not strictly specify them.

### First Embodiment

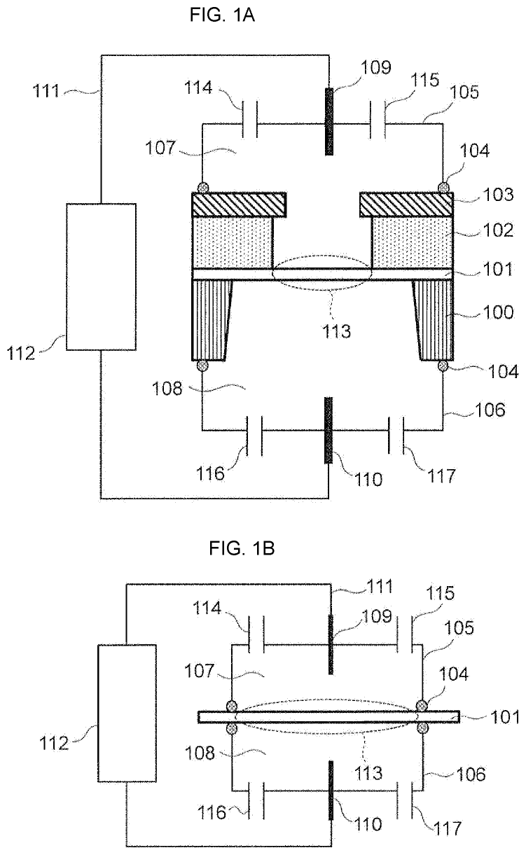

FIG. 1A is a schematic diagram illustrating a configuration of a device for a nanopore forming method according to the first embodiment. As illustrated in FIG. 1A, the device for the nanopore forming method include a Si substrate 100, a SiNx film 101, a SiO₂ film 102, a SiNx film 103, an O-ring 104, a first chamber 105, a second chamber 106, electrodes 109 and 110 (first electrode and second electrode), a wiring 111, and a control unit 112.

The Si substrate 100, the SiNx film 101, the SiO₂ film 102, and the SiNx film 103 are arranged in this order. The O-rings 104 are arranged on an upper surface of the SiNx film 103 and a lower surface of the Si substrate 100, respectively. The first chamber 105 and the second chamber 106 are sealed by these O-rings 104.

The SiNx film 101 has a SiNx membrane 113 (film) as illustrated in the dotted frame in FIG. 1A. The SiNx membrane 113 is a place where a nanopore is formed. The Si substrate 100 and the SiO₂ film 102 do not exist above and below the SiNx membrane 113. An upper surface and a lower surface of the SiNx membrane 113 come into contact with the first aqueous solution 107 and the second aqueous solution 108, respectively.

Of the above structures, the SiO₂ film 102 and the SiNx film 103 are for adjusting a region (size) of the SiNx membrane 113, and are not essential. That is, the SiO₂ film 102 and the SiNx film 103 may or may not be present. The Si substrate 100 also exists as a member that supports the SiNx film 101 and the SiNx membrane 113. Therefore, the Si substrate 100 may be a member made of a material other than Si.

FIG. 1B is a schematic diagram illustrating another configuration of the device for the nanopore forming method. As illustrated in FIG. 1B, without using the Si substrate 100, and without arranging the SiO₂ film 102 and the SiNx film 103, the SiNx film 101 may be directly setup in the first chamber 105 and the second chamber 106. That is, it is sufficient that the first aqueous solution 107 and the second aqueous solution 108 are separated via the target SiNx membrane 113.

The composition ratio x of SiNx membrane 113 is 1<x<4/3. The composition ratio x is an average value of the values measured at a plurality of points in a thickness direction from a front surface or a back surface of the membrane by X-ray photoelectron spectroscopy (XPS) analysis or secondary ion mass spectrometry (SIMS). In the case of FIG. 1A, it may be difficult to measure the composition ratio x if the region of the SiNx membrane 113 is small. In that case, the composition ratio x of the SiNx membrane 113 can be known by measuring the composition ratio x of the region other than a portion of the SiNx membrane 113 of the SiNx film 101. As can be seen from FIGS. 1A and 1B, only a part of the SiNx film 101 is referred to as the SiNx membrane 113, and the SiNx film 101 and the SiNx membrane 113 are a single film which is formed in the same film forming process with the same material. Therefore, in the SiNx film 101, the composition ratio x of the region other than the portion of the SiNx membrane 113 and the composition ratio x of the portion of the SiNx membrane 113 are the same.

A method for measuring the composition ratio x of the SiNx membrane 113, for example, in the case of FIG. 1A, is as follows. The SiO₂ film 102 and the SiNx film 103 above the Si substrate 100 are removed by etching to expose the SiNx film 101 on the Si substrate 100. After that, the composition ratio x at each depth in the film can be measured by measuring XPS every time the SiNx film 101 is etched from the surface in the depth direction. Then, the measured composition ratio x can be considered as the composition ratio of the SiNx membrane 113.

The location where the composition ratio x is measured may be any location on the SiNx film 101 in the horizontal direction. The measurement interval of the SiNx film 101 in the thickness direction (depth direction) can be, for example, 0.2 to 3 nm.

The first chamber 105 is provided with an aqueous solution inlet 114 and an aqueous solution outlet 115, and the first aqueous solution 107 is introduced from the aqueous solution inlet 114. The second chamber 106 is provided with an aqueous solution inlet 116 and an aqueous solution outlet 117, and the second aqueous solution 108 is introduced from the aqueous solution inlet 116. Details of the first aqueous solution 107 and the second aqueous solution 108 will be described later.

The electrode 109 (first electrode) is arranged to come into contact with the first aqueous solution 107 in the first chamber 105. The electrode 110 (second electrode) is arranged to come into contact with the second aqueous solution 108 in the second chamber 106. The electrodes 109 and 110 are connected to the control unit 112 by the wiring 111. The electrodes 109 and 110 are, for example, silver/silver chloride electrodes.

Although not illustrated, the control unit 112 includes a power source for applying a voltage to the electrodes 109 and 110 and an ammeter for measuring the current between the electrodes 109 and 110, and controls them. Further, the control unit 112 controls, for example, to stop the application of the voltage when the current value between the electrodes 109 and 110 reaches a predetermined threshold current. Furthermore, the control unit 112 may include an input unit for setting the threshold current by a user, a storage unit for storing information and measurement results input to the input unit, and a display unit for displaying measurement conditions, measurement results, and the like.

The nanopore forming method according to this embodiment is a method of forming nanopores by a dielectric breakdown method in a similar manner as the methods described in PTL 1 and NPLs 2 to 4 using the setup illustrated in FIG. 1. The dielectric breakdown method is a method of forming a nanopore in the SiNx membrane 113, in which the current flowing between the electrodes 109 and 110 is measured while continuously applying a constant voltage to the electrodes 109 and 110, and it is determined that the nanopore is formed when the current value between the electrodes rises sharply due to the dielectric breakdown of the SiNx membrane 113 and reaches a predetermined threshold current, and the application of voltage is stopped.

In other words, the nanopore forming method of this embodiment includes arranging the SiNx membrane 113 between the first aqueous solution 107 and the second aqueous solution 108, bringing the electrode 109 into contact with the first aqueous solution 107, and bringing the electrode 110 into contact with the second aqueous solution 108, and applying the voltage to the electrodes 109 and 110.

In this embodiment, the pH of the first aqueous solution 107 and the second aqueous solution 108 are 10 or higher, respectively. As the first aqueous solution 107 and the second aqueous solution 108, for example, a KCl aqueous solution can be used. A single nanopore can be formed by using an aqueous solution of such as LiCl, NaCl, CaCl₂), MgCl₂, or CsCl instead of the KCl aqueous solution.

The concentration of the first aqueous solution 107 and the second aqueous solution 108 can be, for example, 1 M, but may be higher than 1 M (for example, 1 M or more and 3M or less) or lower than 1 M (for example, 0.001 M or more and 1 M or less). If the pH is 10 or more (if the film thickness of the SiNx membrane is 20 nm or more, pH is larger than 11), a single nanopore can be formed.

Hereinafter, nanopore formation by the dielectric breakdown method will be described in more detail with experimental contents.

### First Experimental Example: Nanopore Formation by Dielectric Breakdown Method

### First Comparative Example

In a first comparative example, the nanopore formation of nanopores was attempted by the above-mentioned dielectric breakdown method using the setup illustrated in FIG. 1.

Specifically, in the setup illustrated in FIG. 1(a), a SiNx membrane with the thickness of 20 nm and the composition ratio x of 1<x<4/3 was used as the SiNx membrane 113, and the SiO₂ film 102 had a thickness of 250 nm and the SiNx film 103 had a thickness of 100 nm. A KCl aqueous solution having a concentration of 1 M adjusted to pH 7.5 was introduced into the first chamber 105 and the second chamber 106 as the first aqueous solution and the second aqueous solution, respectively.

The predetermined threshold current was set to 0.3×10⁻⁶ A, and the control unit 112 was set to stop applying the voltage to the electrodes 109 and 110 when the current value reached 0.3×10⁻⁶ A. With these settings, the nanopore formation was attempted by applying 0 V to the electrode 109 and 20 V to the electrode 110.

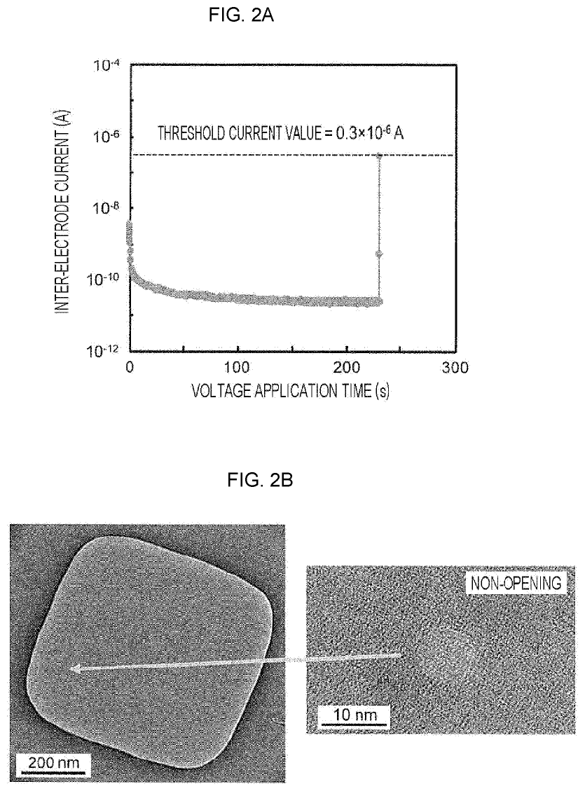

The current value when the voltage was applied was measured, and the TEM observation of the SiNx membrane after dielectric breakdown was performed. The result is illustrated in FIGS. 2A and 2B.

FIG. 2A is a graph illustrating an inter-electrode current value (A) when a voltage is applied in the first comparative example. As illustrated in FIG. 2A, when the voltage application time was about 230 s, the inter-electrode current value rapidly rose and reached 0.3×10⁻⁶ A.

FIG. 2B is a TEM photograph of the SiNx membrane after dielectric breakdown in the first comparative example. The left side of FIG. 2B is a photograph of the entire view of the SiNx membrane seen from the upper surface. Hereinafter, in the TEM photographs of FIGS. 2A and 2B and the like, it is illustrated that the brighter the color, the thinner the film. As indicated by the arrow in the photograph on the left side of FIG. 2B, it can be seen that a part of the SiNx membrane has a bright color. The enlarged photograph of this part is the photograph on the right side of FIG. 2B. Looking at the photograph on the right side of FIG. 2B, it was found that there was no pore and only a thin film region with a diameter of about 10 nm was formed. If there is a pore, the amorphous pattern derived from SiNx is not visible in the perforated region. That is, it was found that when an aqueous solution having a pH of 7.5 was used, a nanopore was not formed even after the SiNx membrane was dielectrically broken down, and the formation of the nanopore was hindered.

### Second Comparative Example

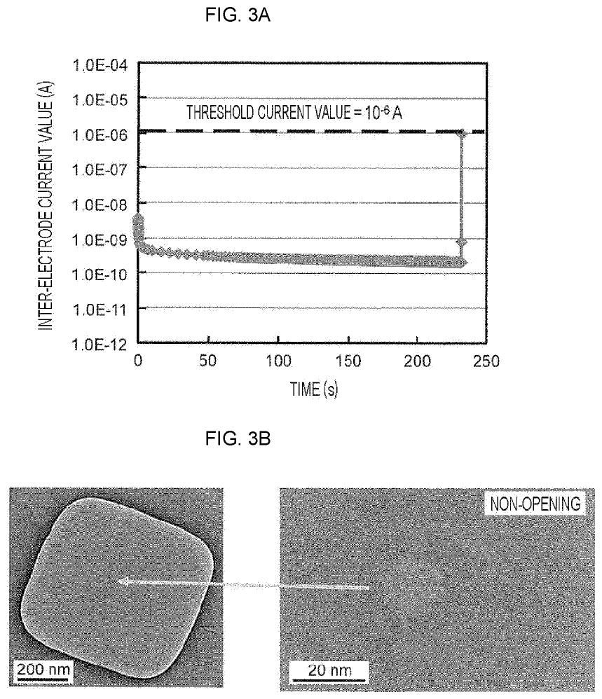

In a second comparative example, an attempt was made for forming a nanopore in the SiNx membrane (1<x<4/3) by the dielectric breakdown method in the same manner as in the first comparative example except that the predetermined threshold current was set to 1×10⁻⁶ A. Further, as in the first comparative example, the current value when the voltage was applied was measured, and the TEM observation of the SiNx membrane after dielectric breakdown was performed. The results are illustrated in FIGS. 3A and 3B.

FIG. 3A is a graph illustrating the current value when a voltage is applied in the second comparative example. As illustrated in FIG. 3A, when the voltage application time was about 240 s, the inter-electrode current value rapidly rose and reached 1×10⁻⁶ A.

FIG. 3B is a TEM photograph of the SiNx membrane after dielectric breakdown in the second comparative example. In the photograph on the left side of FIG. 3B, as indicated by the arrow, it can be seen that a part of the SiNx membrane has a bright color. The enlarged photograph of this part is the photograph on the right side of FIG. 3B. Looking at the photograph on the right side of FIG. 3B, it can be seen that a large thin film region is formed as compared with the case of FIG. 2B, but it is also found that there is no pore. That is, it was found that when an aqueous solution having a pH of 7.5 was used, a nanopore was not formed even after the SiNx membrane had undergone dielectric breakdown.

### Third Comparative Example

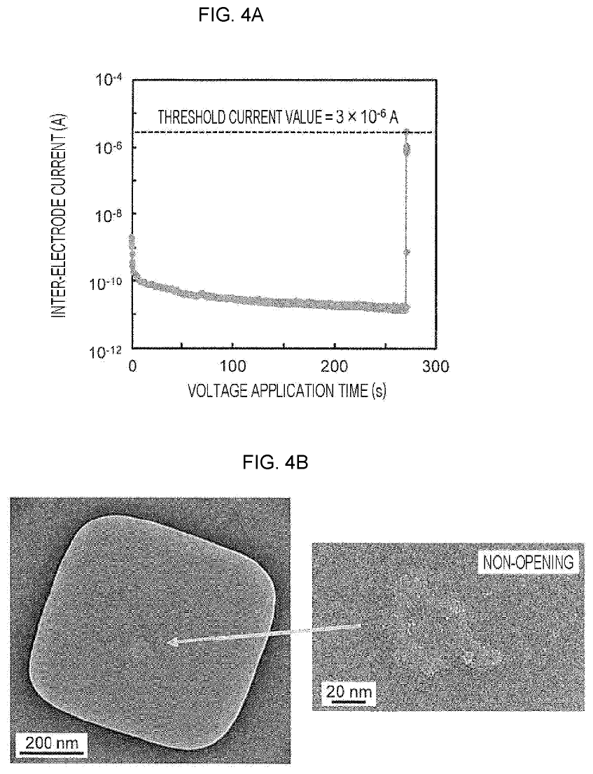

In a third comparative example, an attempt was made for forming a nanopore in the SiNx membrane (1<x<4/3) by the dielectric breakdown method in the same manner as in the first comparative example except that the threshold current was set to 3×10⁻⁶ A. Further, as in the first comparative example, the current value when the voltage was applied was measured, and the TEM observation of the SiNx membrane after dielectric breakdown was performed. The results are illustrated in FIGS. 4A and 4B.

FIG. 4A is a graph illustrating the current value when a voltage is applied in the third comparative example. As illustrated in FIG. 4A, when the voltage application time was about 270 s, the inter-electrode current value rapidly rose and reached 3×10⁻⁶ A.

FIG. 4B is a TEM photograph of the SiNx membrane after dielectric breakdown in the third comparative example. In the photograph on the left side of FIG. 4B, as indicated by the arrow, it can be seen that a part of the SiNx membrane has a bright color. The enlarged photograph of this part is the photograph on the right side of FIG. 4B. Looking at the photograph on the right side of FIG. 4B, it can be seen that a large thin film region is formed as compared with the case of FIG. 3B, but it is also found that there is no pore. That is, it was found that when an aqueous solution having a pH of 7.5 was used, a nanopore was not formed even after the SiNx membrane had undergone dielectric breakdown.

Although not illustrated, the nanopore was not formed as a matter of course even when the threshold current was set to be smaller than 0.3×10⁻⁶ A, for example, 0.1×10⁻⁶ A or less.

### Fourth Comparative Example

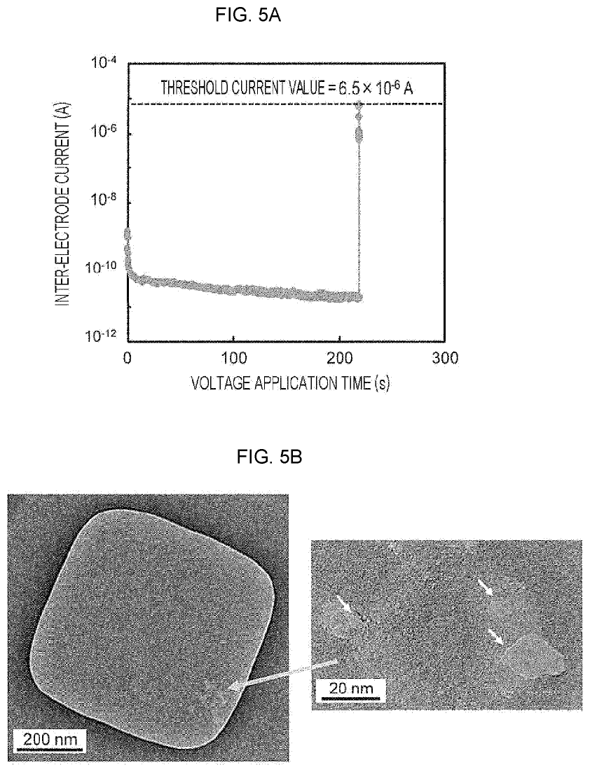

In a fourth comparative example, an attempt was made for forming a nanopore by the dielectric breakdown method in the same manner as in the first comparative example except that the threshold current was set to 6.5×10⁻⁶ A. Further, as in the first comparative example, the current value when the voltage was applied was measured, and the TEM observation of the SiNx membrane after dielectric breakdown was performed. The results are illustrated in FIGS. 5A and 5B.

FIG. 5A is a graph illustrating the current value when a voltage is applied in the fourth comparative example. As illustrated in FIG. 5A, when the voltage application time was about 220 s, the inter-electrode current value rapidly rose and reached 6.5×10⁻⁶ A.

FIG. 5B is a TEM photograph of the SiNx membrane after dielectric breakdown in the fourth comparative example. In the photograph on the left side of FIG. 5B, as indicated by the arrow, it can be seen that a part of the SiNx membrane has a bright color. The enlarged photograph of this part is the photograph on the right side of FIG. 5B.

In the photograph on the right side of FIG. 5B, as indicated by the arrow, it was found that there was a part where the amorphous pattern derived from SiNx could not be seen and there was a part with pores. However, it was found that a plurality of pores having different sizes were formed. When detecting or counting molecules or particles that pass through a nanopore, if multiple nanopores are open on the membrane, the molecules or particles to be detected may enter different nanopores at the same time. In such a case, the change in the ionic current passing through the nanopores is complicated, and as a result, the accuracy of detection or counting of the molecules or particles to be detected is lowered.

Therefore, it is desirable that the number of nanopores present on the membrane is one. Another problem is that pores of various sizes are created. This is because if only pores smaller than the molecules or particles to be detected are formed, the molecule or particle to be detected cannot pass through the pores, and as a result, the molecules or particles to be detected cannot be detected or counted.

As described in the first to fourth comparative examples (FIGS. 2A to 5B), it was found that when the threshold current value is set low, a pore is not opened in the SiNx membrane, and when the threshold current value is set high, pores are opened, but the pores having different sizes are formed. Further, it was found that even if the experiment is performed by setting the threshold current value between the threshold current value (3×10⁻⁶ A) of the third comparative example and the threshold current value (6.5×10⁻⁶ A) of the fourth comparative example, no pore is opened in the SiNx membrane or multiple pores having different sizes are formed. That is, it was found that it is impossible to form a single nanopore on the SiNx membrane simply by applying the methods described in PTL 1 and NPLs 2 to 4 as they are.

On the other hand, PTL 1 and NPLs 2 to 4 describe that a single nanopore can be formed on the SiNx membrane by the method of causing dielectric breakdown as described above. For example, in NPL 2, a KCl aqueous solution having a pH of 10 and a concentration of 1 M is filled on both sides of the SiNx membrane, a voltage is applied to the SiNx membrane, and the threshold current value for stopping the voltage application is set to about 100 nA so as to form a single nanopore with a diameter of about 3 nm. It is also described that a single nanopore can be formed on the SiNx membrane by the same method even when the pH is set to 2, 4, 7, 10 or 13.5.

In NPL 3, a KCl aqueous solution having a pH of 8 and a concentration of 1 M is filled on both sides of the SiNx membrane, a voltage is applied to the SiNx membrane, and the threshold current value for stopping the voltage application is set to about 95 nA so as to form a single nanopore.

In NPL 4, a KCl aqueous solution having a pH of 10 and a concentration of 1 M is filled on both sides of the SiNx membrane, a voltage is applied to the SiNx membrane, and the threshold current value for stopping the voltage application is defined as (threshold current value) (inter-electrode current observed before dielectric breakdown)=½±0.1 so as to form a single nanopore.

On the other hand, as described in the first to fourth comparative examples, we filled a KCl aqueous solution having a pH of 7.5 and a concentration of 1 M on both sides of the SiNx membrane, applied a voltage to the SiNx membrane, and examined a plurality of threshold current values for stopping the voltage application between about 100 nA or less to 10 μA. However, it was impossible to form a single nanopore. Moreover, even if the pH of the KCl aqueous solution having 1 M to be used was set to 7 or 8, the tendency of the above-mentioned experimental results did not change, and it was impossible to form a single nanopore.

As a result of intensive research on the cause of this difference, the following was found. That is, it was found that when the composition ratio x of the SiNx membrane is in the range of 1<x<4/3, a single nanopore cannot be formed by the dielectric breakdown method as described above in an aqueous solution having a pH smaller than 10, and the results illustrated in FIGS. 2A to 5B were obtained.

Similarly, when the composition ratio x of the SiNx membrane is smaller than 1, an attempt was made to form a nanopore by the dielectric breakdown method. As a result, even though an aqueous solution having a pH smaller than 10 was used as the first aqueous solution 107 and the second aqueous solution 108, a single nanopore was formed.

Therefore, a method of forming a single nanopore on the SiNx membrane (1<x<4/3) by the dielectric breakdown method will be described with reference to the first example below. Specifically, in the nanopore forming method according to this embodiment, the pH of the first aqueous solution 107 and the second aqueous solution 108 is set to 10 or more.

### First Example

In the first example, as in the first comparative example, an attempt was made to form a nanopore on a SiNx membrane (1<x<4/3) having a thickness of 20 nm by a dielectric breakdown method using the setup illustrated in FIG. 1A.

In the first example, the composition ratio x of the SiNx membrane was set to about x=1.13, and as the first aqueous solution and the second aqueous solution, a KCl aqueous solution having a concentration of 1 M adjusted to pH 13.1 was introduced to the first chamber 105 and the second chamber 106, respectively. The threshold current was set to 1×10⁻⁶ A. Other conditions were the same as in the first comparative example above, and the nanopore was formed by the dielectric breakdown method.

The composition ratio x was the average value obtained by XPS measurement each time the SiNx film 101 on the Si substrate 100 was etched from the surface in the depth direction after the SiNx film 101 on the Si substrate 100 was exposed. Specifically, ULVAC-PHI's PHI 5000 VersaProbe II (“PHI” is a registered trademark) was used as the XPS device, an X-ray source was Al Kα, and the measurement interval in the depth direction was approximately 0.7 to 0.8 nm. The average value of 26 measurement points was calculated. As a result, x=1.13 was obtained. In this the first example, an example in the case of x=1.13 is described, but as described above, the composition ratio x of the SiNx membrane may be in the range of 1<x<4/3.

Further, as in the first comparative example, the current value when the voltage was applied was measured, and the TEM observation of the SiNx membrane after dielectric breakdown was performed.

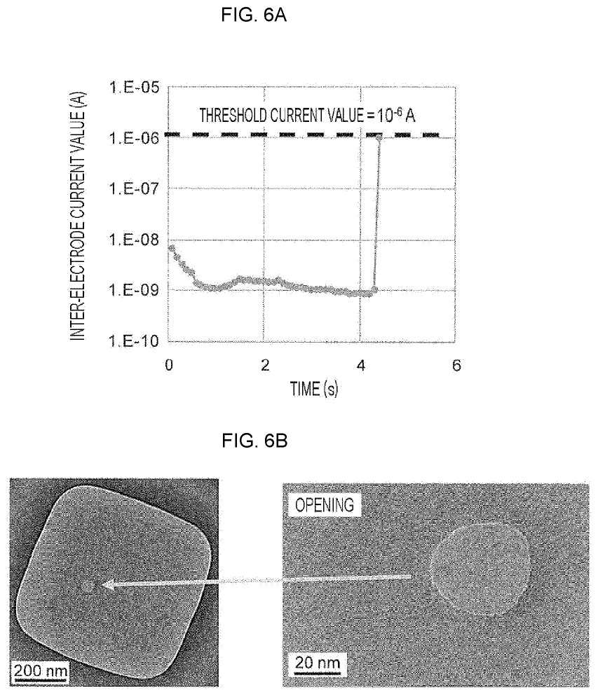

FIG. 6A is a graph illustrating the current value when a voltage is applied in the first example. As illustrated in FIG. 6A, when the voltage application time was about 4 s, the inter-electrode current value rapidly rose and reached 1×10⁻⁶ A.

FIG. 6B is a TEM photograph of the SiNx membrane after dielectric breakdown in the first example. The photograph on the left side of FIG. 6B is a photograph of the entire view of the SiNx membrane seen from the upper surface. In the photograph on the left side of FIG. 6B, as indicated by the arrow, it can be seen that a part of the SiNx membrane has a bright color. The enlarged photograph of this part is the photograph on the right side of FIG. 6B. In the photograph on the right side of FIG. 6B, it was found that there was a part where the amorphous pattern derived from SiNx could not be seen, and the pore (nanopore) was open. Further, from the photograph on the left side of FIG. 6B, it was found that only one pore was formed in the SiNx membrane.

### Second Experimental Example: pH Change

Similar to the first experimental example, an attempt was made to form a nanopore in the SiNx membrane (1<x<4/3) having a thickness of 20 nm by the dielectric breakdown method using the setup of FIG. 1.

In the second experimental example, an attempt was made to form a nanopore by the dielectric breakdown method by setting the threshold current to 1×10⁻⁶ A and changing the pH of the aqueous solution in 7 steps of 1, 3, 7.5, 11, 11.4, 12.5, and 13.1. Other conditions are the same as in the first experimental example.

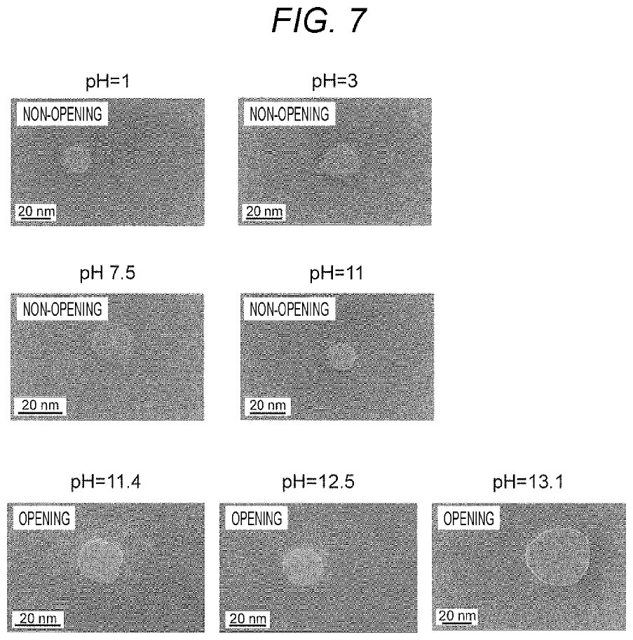

In addition, as in the first experimental example, TEM observation of the SiNx membrane after dielectric breakdown was performed. The results are illustrated in FIG. 7.

FIG. 7 is TEM photographs of the SiNx membranes after dielectric breakdown when the pH of the aqueous solution was changed. As illustrated in FIG. 7, it could be seen that when the pH was higher than 11, there was a portion in the SiNx membrane in which the amorphous pattern could not be seen, and the nanopore was open. Also, one nanopore was formed on each SiNx membrane. On the other hand, when the pH was 11 or less, it was found that only a thin film region was formed in the SiNx membrane and no pores were opened.

As a result of further research, it was found that, by making the thickness of the SiNx membrane (1<x<4/3) thinner than 20 nm, even if the pH of the aqueous solution is in the range of 10 or more and 11 or less, a single nanopore was able to be formed in the SiNx membrane (1<x<4/3) by the dielectric breakdown method.

It was also found that even if the SiNx membrane (1<x<4/3) has a thickness of 20 nm or more, a single nanopore was able to be formed by the dielectric breakdown method in an aqueous solution having a pH higher than 11.

The reason why the pore can be formed in the SiNx membrane (1<x<4/3) in the aqueous solution having a pH of 10 or more (in an aqueous solution having a pH greater than 11 when the thickness of the SiNx membrane is 20 nm or more) by the dielectric breakdown method will be briefly described below.

For example, as in the second comparative example, in an aqueous solution having a pH of 7.5, even when the inter-electrode current reaches the threshold current after dielectric breakdown, no pore is opened and only a local thin film region is formed. It was found through studies that the composition of the film in this local thin film region was extremely higher in Si than in N.

Si is known to be etched with a highly alkaline solution at high temperature. Therefore, when the pore formation is performed to the SiNx membrane (1<x<4/3) by the dielectric breakdown method in an aqueous solution having a pH of 10 or more, a local thin film region composed by more Si than N at an extremely high ratio may be formed, but at the same time, the etching of Si with the aqueous alkaline solution occurs immediately, so that the pore is formed without leaving a local thin film region. Since SiNx (1<x<4/3) is not etched even in an alkaline aqueous solution, the entire membrane is not etched as a result, and the pore can be locally formed. A high temperature is required to quickly etch Si with an alkaline solution, which is covered by the Joule heat of a large current that flows after dielectric breakdown.

As described above, SiNx membranes, particularly membranes having a composition ratio x in the range of 1<x<4/3 are excellent in breakdown withstand voltage (dielectric breakdown withstand voltage) with respect to the applied voltage. In nanopore measurement, the object is measured while applying a voltage to the membrane, so it is desirable that the dielectric breakdown withstand voltage is high. That is, a membrane that is less likely to undergo dielectric breakdown even when the voltage required for measurement is applied for a longer period of time is desirable for nanopore measurement. It is also known that a high voltage is applied to the membrane due to static electricity generated during setup before measurement, and the membrane undergoes dielectric breakdown (NPL 6). In order to prevent dielectric breakdown due to the high voltage derived from static electricity, it is desirable that the dielectric breakdown withstand voltage is high. That is, a membrane that does not easily break down even when a high voltage is applied is desirable.

In fact, the dielectric withstand voltage of the SiNx membrane having a thickness of 30 nm described in NPL 2 and NPL 3 breaks down in about 1000 seconds when 16 V is applied in a KCl aqueous solution having a pH of 7 and a concentration of 1 M. On the other hand, when the SiNx membrane (1<x<4/3) of this embodiment has a thickness of 20 nm, and 16 V is applied in a KCl aqueous solution having a pH of 7 and a concentration of 1 M, it takes much longer than 1000 seconds until dielectric breakdown occurs. That is, although the SiNx membrane (1<x<4/3) of this embodiment is thinner than the SiNx membrane described in NPL 2 and NPL 3, it takes a long time to reach dielectric breakdown when the same voltage is applied. Therefore, it can be said that the SiNx membrane (1<x<4/3) has an extremely high dielectric withstand voltage. From the results of the studies, it was also found that as x of SiNx is smaller than 1, the dielectric withstand voltage decreases (that is, when the same voltage is applied, the time until the dielectric breakdown is shortened).

### Third Experimental Example: Change of Threshold Current

Next, nanopore formation when the threshold current value is changed will be described.

### Second Example

In the second example, as in the first example, an attempt was made to form a nanopore on a SiNx membrane (1<x<4/3) having a thickness of 20 nm by a dielectric breakdown method using the setup illustrated in FIGS. 1A and 1B.

In the second example, as the first aqueous solution and the second aqueous solution, a KCl aqueous solution having a concentration of 1 M adjusted to pH 13.1 was introduced into the first chamber 105 and the second chamber 106, respectively. Further, a predetermined threshold current was set to 1×10⁻⁶ A, and the control unit 112 was set to stop the application of the voltage by the power source when the current value reaches 1×10⁻⁶ A. With these settings, the electrode 109 was applied with 0 V and the electrode 110 was is applied with 18 V. Other conditions are the same as in the first experimental example.

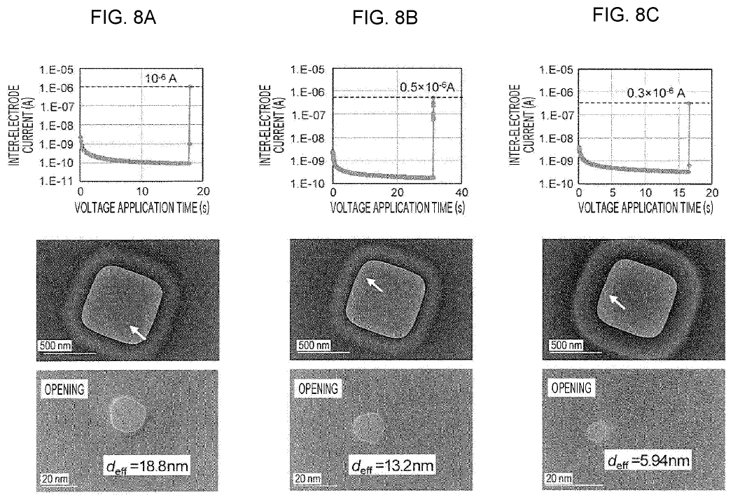

The current value when the voltage was applied was measured, and the TEM observation of the SiNx membrane after dielectric breakdown was performed. Further, assuming that the shape of the formed nanopore is circular, the diameter of the nanopore (effective diameter d_{eff}) was calculated back from the equation of area S=π×(½d_{eff})×(½d_{eff}). The results are illustrated in FIG. 8A.

### Third Example

In the third example, an attempt was made for forming a nanopore by the dielectric breakdown method as in the second example except that the threshold current was set to 0.5×10⁻⁶ A. In addition, the current value when the voltage was applied was measured, and the TEM observation of the SiNx membrane after dielectric breakdown was performed. The effective diameter d_{eff} of the nanopore was determined in the same manner as in the second example. The results are illustrated in FIG. 8B.

### Fourth Embodiment

In the fourth embodiment, an attempt was made for forming a nanopore by the dielectric breakdown method as in the second example except that the threshold current was set to 0.3×10⁻⁶ A. In addition, the current value when the voltage was applied was measured, and the TEM observation of the SiNx membrane after dielectric breakdown was performed. The effective diameter d_{eff} of the nanopore was determined in the same manner as in the second example. The results are illustrated in FIG. 8C.

The upper parts of FIGS. 8A to 8C are graphs illustrating the current value when a voltage is applied, the middle parts are TEM photographs illustrating a panoramic view of the SiNx membrane after dielectric breakdown, and the lower parts are enlarged photograph of the formed nanopore.

As illustrated in the lower parts of FIGS. 8A to 8C, the effective diameter d_{eff} of the nanopore formed in the second example was 18.8 nm, the effective diameter d_{eff} of the nanopore formed in the third example was 13.2 nm, and the effective diameter d_{eff} of the nanopore formed in the fourth embodiment was 5.94 nm. In this way, the size of the formed nanopore can be adjusted by adjusting the current threshold value for stopping the voltage application. From the results of the studies, it was found that the effective diameter d_{eff} of the nanopore can be formed from about 1 nm to 200 nm or more by adjusting the current threshold by the method of this embodiment. It was also found that even if the effective diameter d_{eff} was increased, only a single nanopore was formed on the SiNx membrane.

As described above, in the first to third examples, the KCl aqueous solution was used as the first aqueous solution 107 and the second aqueous solution 108. However, a single nanopore can be formed by using an aqueous solution of such as LiCl, NaCl, CaCl₂), MgCl₂, or CsCl instead of the KCl aqueous solution.

As in the first to third examples, the concentration of the first aqueous solution 107 and the second aqueous solution 108 can be, for example, 1M, but may be higher than 1M (for example, 1M or more and 3M or less) or lower than 1M (for example, 0.001 M or more and 1M or less). If the pH is 10 or more (if the film thickness of the SiNx membrane is 20 nm or more, pH is larger than 11), a single nanopore can be formed.

In this embodiment, a SiNx membrane (1<x<4/3) having a high dielectric withstand voltage is used. Of the SiNx membranes (1<x<4/3), those with a film density in a range of 2.8 g/cm³ or more and 3.2 g/cm³ or less is particular has a high dielectric withstand voltage and suitable for nanopore measurement. Of course, if the pH of the aqueous solution is 10 or more (a value larger than a pH of 11 if the film thickness of the SiNx membrane is 20 nm or more) even though the film density of the SiNx membrane (1<x<4/3) is outside the range of 2.8 g/cm³ or more and 3.2 g/cm³ or less, a single nanopore can be formed by a method in which the current flowing between the electrodes is measured while continuously applying a constant voltage to the membrane, and the current value between the electrodes is increased (that is, the film is subjected to dielectric breakdown) and reaches a preset threshold current, and then the application of the voltage is stopped.

Of the SiNx membranes (1<x<4/3) with high dielectric withstand voltage, those with an etching rate of 0.5 nm/min or less due to hydrofluoric acid diluted to 1/200 have a particularly high dielectric withstand voltage and are suitable for nanopore measurement. Of course, if the pH of the aqueous solution is 10 or more (a value larger than a pH of 11 if the film thickness of the SiNx membrane (1<x<4/3) is 20 nm or more) even though the etching rate with hydrofluoric acid diluted to 1/200 is 0.5 nm/min or more, a single nanopore can be formed by the dielectric breakdown method.

The SiNx membrane (1<x<4/3) with a high dielectric withstand voltage usually has a tensile stress of 600 MPa or more. Since the SiNx membrane used in NPL 2 is described to have a tensile stress of 250 MPa or less, the composition ratio x of SiNx is considered to be smaller than 1.

The film thickness of the SiNx membrane 113 can be 5 nm or more and 100 nm or less. The dielectric breakdown withstand voltage decreases as the film thickness of the SiNx membrane 113 decreases. When the film thickness is less than 5 nm, the rate of decrease in the dielectric breakdown withstand voltage of the SiNx membrane 113 increases with respect to the decrease in film thickness. As a result, there is a risk of interfering with nanopore measurement. Further, when the film thickness of the SiNx membrane 113 is larger than 100 nm, the voltage required for dielectric breakdown exceeds 100 V, so that extremely large Joule heat is generated at the same time as the dielectric breakdown, and the deterioration of the SiNx membrane 113 is remarkable.

Here, NPL 7 discloses a nanopore forming method in which 20 V is applied to the SiNx membrane in the KCl aqueous solution having a pH of 7.5 and a concentration of 1 M, and the voltage application is stopped when the current flowing between the electrodes reaches 10⁻⁶ A, in which a locally thin film region is formed. NPL 7 discloses, as a second procedure, applying a pulse voltage of 10 V and measuring a current while applying a voltage of 0.1 V are repeatedly performed so as to penetrate the locally thin film region. Compared with this embodiment, the method of NPL 7 takes a longer time to form a nanopore due to the presence of the second procedure. In addition, NPL 7 describes that there is a large variation in the d_{eff} of a nanopore when experiments are performed multiple times under the same condition, and d_{eff}=0 (that is, when a nanopore is not formed) also exists.

As a result of attempting nanopore formation by the method described in NPL 7, it was found that it is difficult to form a single nanopore when the d_{eff} is outside the range of about 20 nm to 30 nm. Therefore, the nanopore forming method of this embodiment takes less time to form the nanopore than the method described in NPL 7, and in this embodiment, the size of the formed nanopore varies less, and an adjustment range of the size of a formable nanopore is also wide.

As described above, according to the nanopore forming method of this embodiment, a voltage is applied to the SiNx membrane (1<x<4/3) in the aqueous solution having a pH of 10 or more (a voltage is applied in the aqueous solution having a pH larger than 11 if the film thickness of the SiNx membrane is 20 nm or more), and the dielectric breakdown occurs so that a single nanopore can be stably formed on the SiNx membrane (1<x<4/3) which has a high dielectric withstand voltage and is suitable for nanopore measurement.

### Second Embodiment

Next, a nanopore forming method according to the second embodiment will be described. This embodiment is different from the first embodiment in that a laminated film in which another film is laminated on the SiNx film 101 of FIGS. 1A and 1B is used.

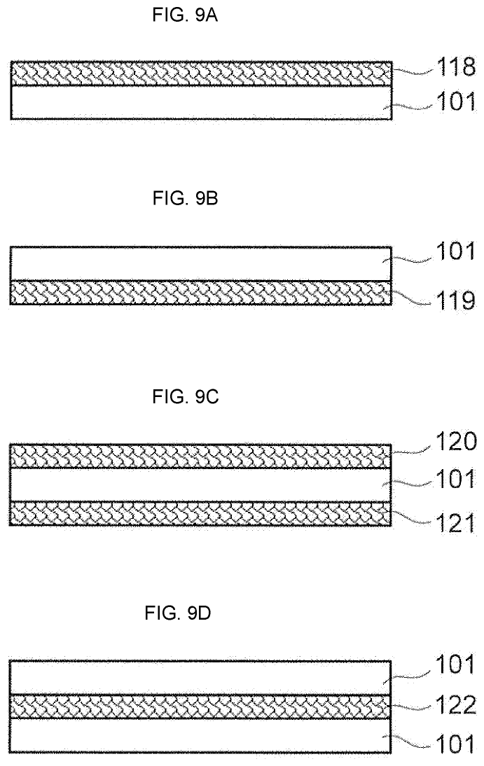

FIGS. 9A-9D are schematic diagrams illustrating the structure of the laminated film according to the second embodiment. In the case of FIG. 9A, a film 118 is laminated on the upper surface of the SiNx film 101 (1<x<4/3). In the case of FIG. 9B, a film 119 is laminated on the lower surface of the SiNx film 101. In the case of FIG. 9C, films 120 and 121 are laminated on both sides of the SiNx film 101. In the case of FIG. 9D, a film 122 is arranged between the two SiNx films 101.

The films 118 to 122 are, for example, SiO₂ film, HfO₂ film, Al₂O₃ film, HfAlOₓ film, ZrAlOx film, Ta₂O₅ film, SiC film, SiCN film, carbon film, a SiNx film having a composition ratio x outside the range of 1<x<4/3, or a composite of these. Further, the films 118 to 121 may be, for example, HMDS films for the purpose of preventing non-specific adsorption of the object to be measured on the surface.

In this way, even when the SiNx membrane (1<x<4/3) and other membranes are laminated, if the pH is less than 10, a single nanopore cannot be formed in the membrane by the above-mentioned dielectric breakdown method. However, if the pH of the aqueous solution is 10 or more (a value larger than pH 11 when the film thickness of the SiNx membrane 113 is 20 nm or more), a single nanopore can be formed in the laminated film containing the SiNx film (1<x<4/3).

### Third Embodiment

Next, a single nanopore forming method according to the third embodiment will be described. This embodiment differs from the first embodiment in that the voltage applied between the electrodes 109 and 110 is not constant but changes with time.

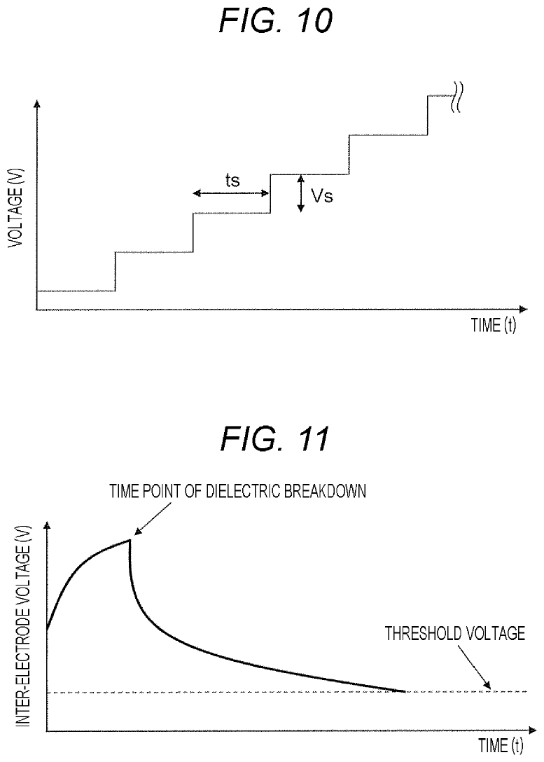

FIG. 10 is a schematic diagram illustrating an example of voltage application in the third embodiment. In the example illustrated in FIG. 10, the control unit 112 controls the power source (not illustrated) so that the voltage applied between the electrodes 109 and 110 increases stepwise. In FIG. 10, tₛ indicates the application time of a certain value of voltage, and Vₛ indicates the amount of increase in voltage.

According to the nanopore forming method according to this embodiment, the time until a nanopore is formed can be shortened by, for example, shortening tₛ or increasing Vₛ. Further, the nanopore forming method according to this embodiment is effective when it is unknown how much voltage and how many seconds the membrane to be used causes dielectric breakdown. The size of the nanopore can be adjusted by adjusting the threshold current for cutting off the applied voltage.

### Fourth Embodiment

Next, a single nanopore forming method according to the fourth embodiment will be described. In this embodiment, the control unit 112 further includes a voltage meter (not illustrated) for measuring the voltage between the electrodes 109 and 110. The nanopore forming method of this embodiment is different from the first embodiment in that the voltage between the electrodes 109 and 110 is measured while controlling the voltage so that the current flowing between the electrodes 109 and 110 is constant, and after the dielectric breakdown, the current supply between the electrodes 109 and 110 is stopped when the voltage between the electrodes 109 and 110 reaches a predetermined threshold voltage.

FIG. 11 is a schematic diagram illustrating an example of voltage application in the fourth embodiment. As illustrated in FIG. 11, the control unit 112 measures the voltage value while applying a voltage between the electrodes 109 and 110 so that the current flowing between the electrodes 109 and 110 is maintained to be constant. The control unit 112 stops supplying the current between the electrodes 109 and 110 when the voltage between the electrodes drops steeply after dielectric breakdown and reaches a predetermined threshold voltage. A single nanopore can also be formed on the SiNx membrane 113 by such a method.

### Fifth Embodiment

Next, a nanopore forming method according to the fifth embodiment will be described. This embodiment is different from the first embodiment in that only one of the first aqueous solution 107 and the second aqueous solution 108 has a pH of 10 or more (a value larger than a pH of 11 when the thickness of the SiNx membrane 113 is 20 nm or more), and the other one has a pH of less than 10. Also in such a configuration, a single nanopore can be formed on the SiNx membrane 113 by the dielectric breakdown method.

In this embodiment, the pH of the aqueous solution on the side in contact with the electrode serving as the negative electrode can be set to 10 or more. For example, a KCl aqueous solution having a concentration of 1 M adjusted to pH 7.5 can be introduced into the first chamber 105, and a KCl aqueous solution having a concentration of 1 M adjusted to pH 13 can be introduced into the second chamber 106. Then, the voltage can be applied to the SiNx membrane 113 to cause a potential difference between the electrodes 109 and 110. At this time, if the electrode 109 is set to have a high potential with respect to the electrode 110 (for example, the electrode 109 has a potential of 20 V and the electrode 110 has a potential of 0 V), the time until the nanopore is formed is shorter compared to a case where the electrode 109 is set to have a low potential with respect to the electrode 110 (for example, the electrode 109 is −20 V and the electrode 110 is 0 V).

The reason is described below. First, it is considered that OH⁻ in the aqueous solution moves toward the positive electrode side by applying a voltage, and the collision of OH⁻ to the SiNx membrane 113 contributes to the formation of the nanopore. Since an aqueous solution having a high pH contains a large amount of OH⁻, it is possible to increase the amount of OH⁻ toward the positive electrode side, that is, toward the SiNx membrane 113 by applying a voltage by arranging an aqueous solution having a high pH on the negative electrode side. In this way, the time until the nanopore is formed is shortened.

As described above, in the nanopore forming method of this embodiment, either one of the first aqueous solution 107 and the second aqueous solution 108 has a pH of 10 or more, and the other aqueous solution has a pH of less than 10. By having such a configuration, this embodiment can shorten the time until the nanopore is formed.

Further, as described above, if the pH of the first aqueous solution 107 and the pH of the second aqueous solution 108 are smaller than 10, especially if the pH is 7 or more and 8 or less, the SiNx membrane having a composition ratio x of 1<x<4/3 has the property that a nanopore is not formed by the dielectric breakdown method as described above. However, as in this embodiment, by setting either one of the first aqueous solution 107 and the second aqueous solution 108 to have a pH of 10 or higher, a single nanopore can be stably formed.

### Sixth Embodiment

In the sixth embodiment, an analysis method using the nanopore formed by the nanopore forming methods described in the first to fifth embodiments will be described.

First, a nanopore is formed by any of the methods of the first to fifth embodiments, and then either the first aqueous solution 107 or the second aqueous solution 108 is replaced with an aqueous solution containing an object to be measured. After that, the current value is measured while applying a voltage between the electrodes 109 and 110. During the measuring, the current value changes significantly when the object to be measured passes through the nanopore.

By counting the number of changes in the current value, it is possible to count the objects to be measured contained in the aqueous solution. In addition, by observing a state of the change in the current value, the structural and electrical characteristics of the object to be measured can be grasped.

If the object to be measured contains DNA having a double helix structure, the structure starts to be dissociated when the pH of the aqueous solution is 9.5 or higher. In addition, DNA starts decomposing in an acidic aqueous solution. Therefore, when the object to be measured contains DNA having a double helix structure, it is desirable that the pH of the aqueous solution containing the object to be measured is 9 or less, and the pH may be in the range of 7 to 8 depending on the situation. Therefore, after forming the nanopore by the methods of the first to fifth embodiments, the first aqueous solution 107 or the second aqueous solution 108 having a pH of 10 or more is replaced with an aqueous solution having a pH of 7 to 9 or a pH of 7 to 8, and then the object to be measured can be introduced into the aqueous solution.

When the nanopore is formed by the methods of the first to fourth embodiments, the object to be measured is placed in the first aqueous solution 107 or the second aqueous solution 108 in the first chamber 105 or the second chamber 106 in advance. By doing so, after the nanopore is formed, the nanopore measurement can be performed without replacing the aqueous solution in the first chamber 105 or the second chamber 106, and the time from the formation of the nanopore to the end of the nanopore measurement can be shortened.

Further, when the nanopore is formed by the method of the fifth embodiment, the pH of one of the aqueous solutions of the first chamber 105 or the second chamber 106 is adjusted to 7 to 9, and 7 to 8 depending on the situation. By doing so, even if the object to be measured containing DNA having a double helix structure is placed therein in advance, the DNA will not be dissociated. Therefore, after the nanopore is formed, the nanopore measurement can be performed without replacing the aqueous solution in the first chamber 105 or the second chamber 106, and the time from the formation of the nanopore to the end of the nanopore measurement can be shortened.

### MODIFICATIONS

The present disclosure is not limited to the examples described above, but includes various modifications. For example, the above examples have been described in detail for easy understanding of the present disclosure, and the present disclosure is not necessarily limited to having all the configurations described. In addition, a part of a certain embodiment can be replaced with the configuration of the other embodiment. Further, it is possible to add the configuration of one embodiment to the configuration of another embodiment. It is also possible to add, delete, or replace a part of the configuration of another example with respect to a part of the configuration of each example.

### REFERENCE SIGNS LIST

100 Si substrate
101 SiNx film
102 SiO₂ film
103 SiNx film
104 O-ring
105 first chamber
106 second chamber
107 first aqueous solution
108 second aqueous solution
109,110 electrode
111 wiring
112 control unit
113 SiNx membrane
114,116 aqueous solution inlet
115,117 aqueous solution outlet
118˜122 film

1. A nanopore forming method, comprising:
arranging a SiNx film between a first aqueous solution and a second aqueous solution;
bringing a first electrode to come into contact with the first aqueous solution and bringing a second electrode to come into contact with the second aqueous solution; and
applying a voltage to the first electrode and the second electrode,
wherein the SiNx film has a composition ratio of 1<x<4/3, and
at least any one of the first aqueous solution and the second aqueous solution has a pH of 10 or more.

2. The nanopore forming method of claim 1, wherein at least any one of the first aqueous solution and the second aqueous solution has a pH larger than 11.

3. The nanopore forming method of claim 1, further comprising stopping an application of the voltage when a current flowing between the first electrode and the second electrode reaches a predetermined threshold current.

4. The nanopore forming method of claim 1, wherein a film density of the SiNx film is 2.8 g/cm³ or more and 3.2 g/cm³ or less.

5. The nanopore forming method of claim 1, wherein a stress of the SiNx film is 600 Mpa or more.

6. The nanopore forming method of claim 1, wherein the SiNx film has an etching rate of 0.5 nm/min or less by an HF aqueous solution diluted to 1/200.

7. The nanopore forming method of claim 1, wherein a film thickness of the SiNx film is 5 nm or more and 100 nm or less.

8. The nanopore forming method of claim 1, wherein at least one of KCl, LiCl, NaCl, CaCl₂, MgCl₂, and CsCl is dissolved in the first aqueous solution and the second aqueous solution.

9. The nanopore forming method of claim 1, wherein, when the first aqueous solution has a pH of 10 or more and the second aqueous solution has a pH of less than 10, the voltage is applied to the first electrode and the second electrode such that a potential of the first aqueous solution is lower than a potential of the second aqueous solution.

10. The nanopore forming method of claim 1, wherein a film different from the SiNx film is laminated on a surface or an inside of the SiNx film.

11. An analysis method of an object to be measured using a nanopore, comprising:
arranging a SiNx film between a first aqueous solution and a second aqueous solution;
bringing a first electrode to come into contact with the first aqueous solution and bringing a second electrode to come into contact with the second aqueous solution;
making the first aqueous solution or the second aqueous solution to contain the object to be measured;
forming the nanopore in the SiNx film by applying a voltage to the first electrode and the second electrode; and
analyzing the object to be measured by measuring a current value between the first electrode and the second electrode when the object to be measured passes through the nanopore,
wherein the SiNx film has a composition ratio of 1<x<4/3, and
at least any one of the first aqueous solution and the second aqueous solution has a pH of 10 or more.

12. The analysis method of claim 11, wherein, when the first aqueous solution has a pH of 10 or more and the second aqueous solution has a pH of less than 10, the object to be measured is contained in the second aqueous solution.

13. An analysis method of an object to be measured using a nanopore, comprising:
arranging a SiNx film between a first aqueous solution and a second aqueous solution;
bringing a first electrode to come into contact with the first aqueous solution and bringing a second electrode to come into contact with the second aqueous solution;
forming the nanopore in the SiNx film by applying a voltage to the first electrode and the second electrode;
replacing at least one of the first aqueous solution and the second aqueous solution after forming the nanopore into an aqueous solution containing the object to be measured; and
analyzing the object to be measured by measuring a current value between the first electrode and the second electrode when the object to be measured passes through the nanopore,
wherein the SiNx film has a composition ratio of 1<x<4/3, and
at least any one of the first aqueous solution and the second aqueous solution has a pH of 10 or more.

14. A nanopore forming method, comprising:
arranging a film between a first aqueous solution and a second aqueous solution;
bringing a first electrode to come into contact with the first aqueous solution and bringing a second electrode to come into contact with the second aqueous solution;
applying a voltage to the first electrode and the second electrode: and
stopping an application of the voltage when a current flowing between the first electrode and the second electrode reaches a predetermined threshold current,
wherein the film has a property that a single nanopore is not formed when a pH of the first aqueous solution and a pH of the second aqueous solution are 7 or more and 8 or less, and
at least any one of the first aqueous solution and the second aqueous solution has a pH of 10 or more when the nanopore is formed.

15. The nanopore forming method of claim 14, wherein the film contains a SiNx film.
